(19) United States
(12) Patent Application Publication
(10) Pub. No.: US 2018/0099112 A1
(43) Pub. Date: Apr. 12, 2018

(54) METHODS AND DEVICES FOR SUPPORTING AN ENDOTRACHEAL TUBE

(71) Applicant: THE HEALTH CORPORATION OF THE GALILEE MEDICAL CENTER, Nahariya (IL)

(72) Inventor: ARKADIY BELENKIY, MAALOT (IL)

(21) Appl. No.: 15/835,770

(22) Filed: Dec. 8, 2017

Related U.S. Application Data
(63) Continuation-in-part of application No. PCT/IB2016/053349, filed on Jun. 8, 2016.
(60) Provisional application No. 62/172,269, filed on Jun. 8, 2015.

(51) Int. Cl. A61M 16/04; A61M 16/06

(52) U.S. Cl. CPC A61M 16/0488; A61M 16/0683; A61M 25/02; A61M 16/0463; A61M 16/0484; A61M 16/0402

(57) ABSTRACT

An orotracheal tube holder comprises an elongated body having a distal end and a proximal end. The distal end is bite resistant and dimensioned to pass into the mouth of a human. The elongated body defines a void not less than 5 cm long with an axis passing through the void. The elongated body is configured to contain an orotracheal tube inside the void.

### CROSS-REFERENCE TO RELATED APPLICATIONS

The present application is a continuation-in-part of PCT Patent Application No. PCT/M2016/053349, filed Jun. 8, 2016, which in turn is based upon and claims the priority of U.S. Provisional Patent Application Ser. No. 62/172,269, filed Jun. 8, 2015, each of which is incorporated herein in its entirety.

### FIELD OF THE INVENTION

The invention, in some embodiments, relates to the field of medicine, and more specifically, to methods and devices for supporting an orotracheal tube.

### BACKGROUND OF THE INVENTION

Tracheal intubation, the deployment of an artificial tube in the trachea of a subject to ensure an open airway to allow breathing, is known in the medical arts.

A particular type of tracheal intubation is orotracheal intubation, where an orotracheal tube is deployed to pass into and through the mouth, through the larynx and to the trachea.

Methods and devices for supporting orotracheal tubes have been described in the art, including in U.S. Pat. Nos. 3,946,742 and U.S. 5,437,273.

### SUMMARY OF THE INVENTION

Some embodiments of the invention relate to methods and devices for supporting an orotracheal tube, especially when deployed in a human.

According to an aspect of some embodiments of the teachings herein, there is provided an orotracheal tube holder harness (10) configured to be worn by a human, comprising:
a strap assembly (12) including a chin portion (14), the strap assembly (12) configured for encircling the chin and head and/or neck of a human wearing the harness (10);
a connector element (16) movably attached to the strap assembly (12) through a proximal portion (18) thereof, so that the connector element (16) extends from the chin portion (14) of the strap assembly (12); and
a distal portion (20) of the connector element (16) including an orotracheal tube holder attachment (22), configured for attachment of an elongated orotracheal tube holder (24) having an axis (26),
wherein, the axis (26) of a tube holder (24) attached to the tube holder attachment (22) of the connector element (18) is directable at a mouth of a human wearing the harness (10). In some embodiments, the harness further comprises an elongated orotracheal tube holder (24) attached to the tube holder attachment (22), the orotracheal tube holder having a distal end (44), a proximal end (46) and an axis (26), the axis directable at the mouth of a human wearing the harness. In some embodiments, the axis is directable through the mouth towards the cervical vertebrae of a human wearing the harness.

According to an aspect of some embodiments of the teachings herein, there is also provided an orotracheal tube holder (24), comprising:
an elongated body (48) having a distal end (44) and a proximal end (46);
the distal end (44) being bite resistant and dimensioned to pass into the mouth of a human;
the body defining a void (50) not less than 5 cm long with an axis (26) passing through the void,
the body configured for containing an orotracheal tube (52) inside the void.

According to an aspect of some embodiments of the teachings herein, there is also provided a kit, comprising:
an orotracheal tube holder harness (10) according to the teachings herein; and
an orotracheal tube holder (24) according to the teachings herein. In some embodiments, the harness and the tube holder are provided in the kit as physically separated components that can be optionally physically associated for use. In some embodiments, the harness and the tube holder are provided in the kit physically associated for use.

Unless otherwise defined, all technical and scientific terms used herein have the same meaning as commonly understood by one of ordinary skill in the art to which the invention pertains. In case of conflict, the specification, including definitions, will take precedence.

As used herein, the terms “comprising”, “including”, “having” and grammatical variants thereof are to be taken as specifying the stated features, integers, steps or components but do not preclude the addition of one or more additional features, integers, steps, components or groups thereof. These terms encompass the terms “consisting of” and “consisting essentially of”.

As used herein, the indefinite articles “a” and “an” mean “at least one” or “one or more” unless the context clearly dictates otherwise.

As used herein, when a numerical value is preceded by the term “about”, the term “about” is intended to indicate +/−10%.

### BRIEF DESCRIPTION OF THE DRAWINGS

Some embodiments of the invention are described herein with reference to the accompanying figures. The description, together with the figures, makes apparent to a person having ordinary skill in the art how some embodiments of the invention may be practiced. The figures are for the purpose of illustrative discussion and no attempt is made to show structural details of an embodiment in more detail than is necessary for a fundamental understanding of the invention. For the sake of clarity, some objects depicted in the figures are not to scale.

In the Figures:

FIG. 1 schematically depicts an embodiment of an orotracheal tube holder harness and an orotracheal tube holder according to an embodiment of the teachings herein supporting an orotracheal tube for use with a human subject, in side view; and

FIGS. 2A, 2B, 2C, 2D and 2E schematically depict an orotracheal tube holder according to an embodiment of the teachings herein.

### DESCRIPTION OF SOME EMBODIMENTS OF THE INVENTION

Some embodiments of the invention relate to methods and devices for supporting an orotracheal tube.

When used for holding an orotracheal tube in a human, some embodiments of the teachings herein provide one or more of: leaving the philtrum free of obstruction; use on a person having a mustache and/or beard without requiring shaving thereof; and/or leaving an space of at least 3 cm (in some embodiments more) from the lower lip free of obstruction.

Before explaining at least one embodiment in detail, it is to be understood that the invention is not necessarily limited in its application to the details of construction and the arrangement of the components and/or methods set forth herein. The invention is capable of other embodiments or of being practiced or carried out in various ways. The phraseology and terminology employed herein are for descriptive purpose and should not be regarded as limiting.

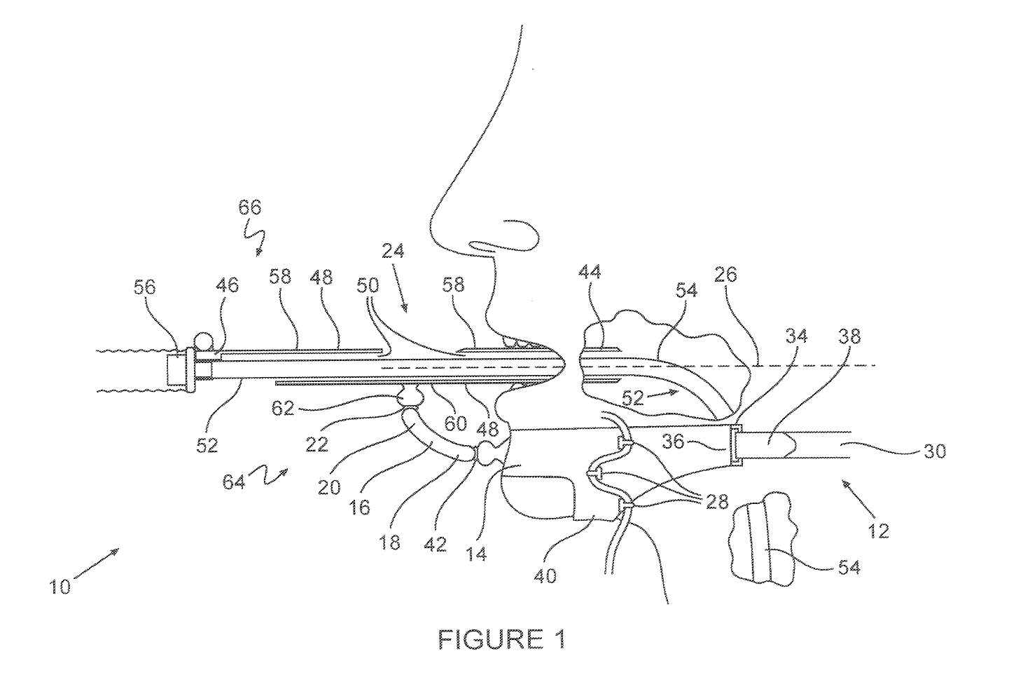

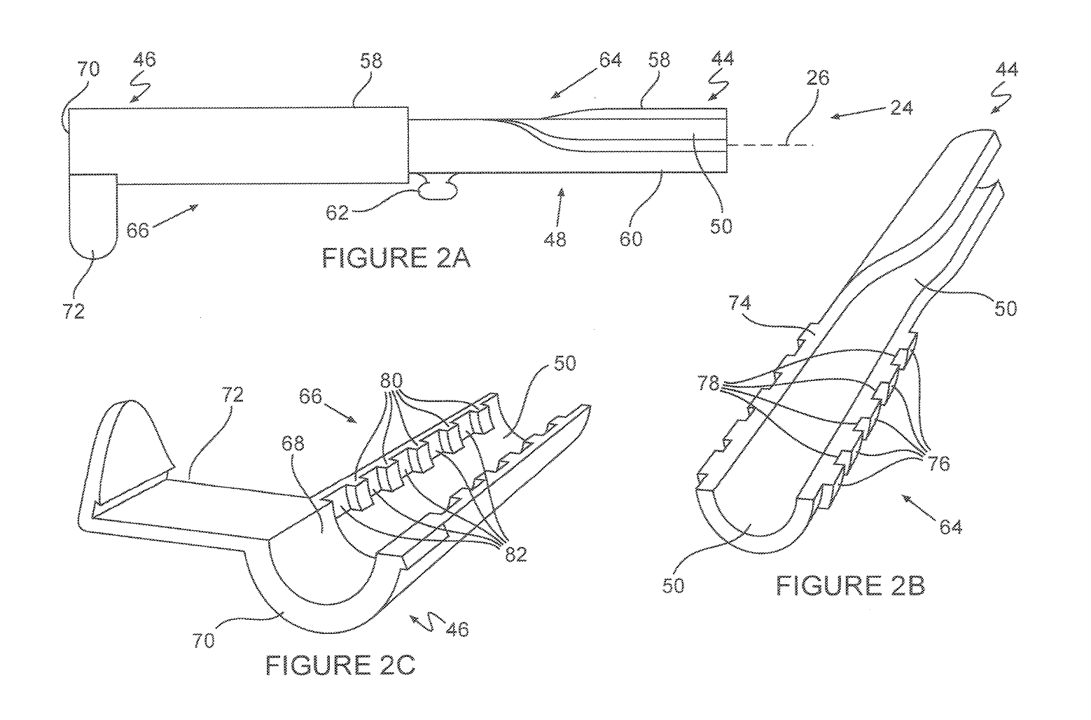

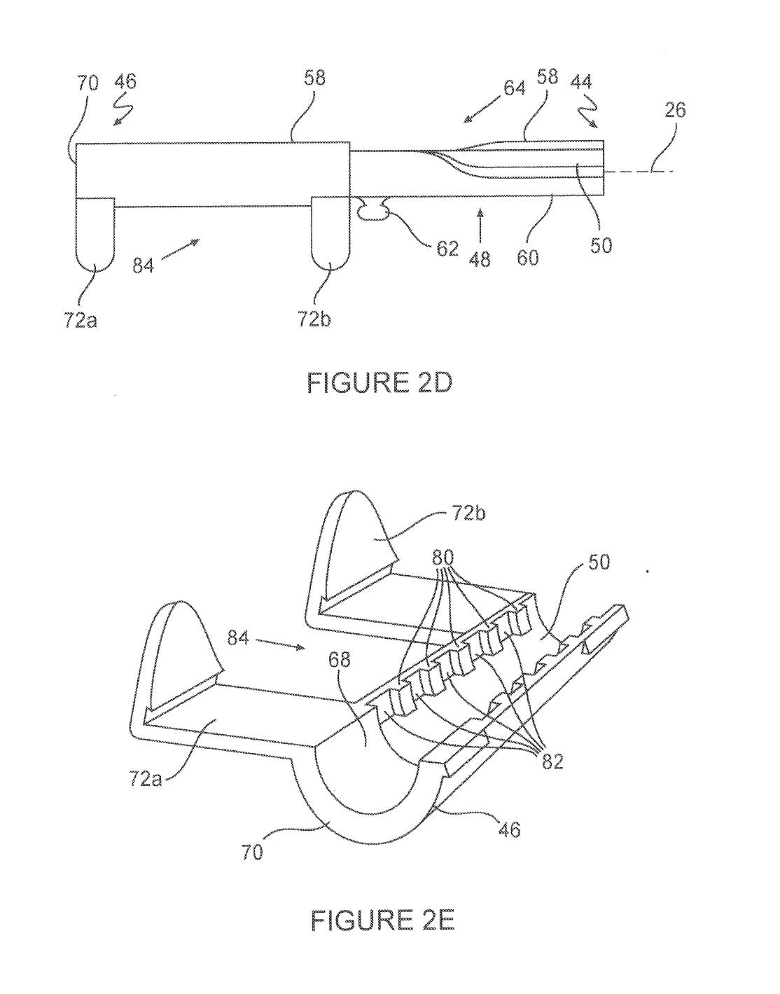

Embodiments of an orotracheal tube holder harness and an orotracheal tube holder according to the teachings herein are described with reference to FIGS. 1 and 2A-2E.

According to an aspect of some embodiments of the teachings herein, there is provided an orotracheal tube holder harness (10) configured to be worn by a human, comprising:
a strap assembly (12) including a chin portion (14), the strap assembly (12) configured for encircling the chin and head and/or neck of a human wearing the harness (10);
a connector element (16) movably attached to the strap assembly (12) through a proximal portion (18) thereof, so that the connector element (16) extends from the chin portion (14) of the strap assembly (12); and
a distal portion (20) of the connector element (16) including an orotracheal tube holder attachment (22), configured for attachment of an elongated orotracheal tube holder (24) having an axis (26),
wherein, the axis (26) of a tube holder (24) attached to the tube holder attachment (22) of the connector element (18) is directable at a mouth of a human wearing the harness (10).

As used herein, an elongated orotracheal tube holder is a device configured to hold an orotracheal tube, such holding effective to prevent axial slipping of the orotracheal tube in the tube holder when the orotracheal tube is deployed for use in a human.

The chin portion (e.g., 14) of the strap assembly (e.g., 12) is a portion that, when the harness is worn by a human, is located in front of the front of the chin of the human.

In some embodiments, the axis (e.g., 26) of a tube holder attached to the tube holder attachment of the connector element is directable through the mouth towards the cervical vertebrae of the human wearing the harness, as depicted in FIG. 1.

In some embodiments, a harness further comprises a nasogastric feeding tube holder (28) functionally associated with the strap assembly.

In some embodiments, the strap assembly includes an encircling band (30) functionally associated with chin portion (14) of the strap assembly (12), together configured to encircle the front of the chin and at least a portion of the cervical spine, in some embodiments somewhere over c1-c4, in some embodiments c1-c3, and in some embodiments somewhere over c2-c3 of a human subject wearing the harness. In some such embodiments, the encircling band is configured to have an adjustable diameter to comfortably fit on different sized/shaped human heads. In some such embodiments, the encircling band is at least partially elastic. In some such embodiments, the encircling band is a continuous band. In some such embodiments, the encircling band is a discontinuous band configured to be opened and closed, e.g., with a hook and loop fastener or a buckle.

In some embodiments, the strap assembly is configured to allow reversible separation of the strap assembly into at least two parts while an orotracheal tube is held in place in a human subject with the harness, a first of the at least two parts including the chin portion and a second of the at least two parts including other portions of the strap assembly. For example, in harness (10) depicted in FIG. 1, strap assembly (12) includes two parts, the first part including chin portion (14) and the second part including an encircling band (30). The first part includes two fastening loops (34, only one visible) at each of the two ends (36) of the first part, while the two ends of the second part (only one visible) constitute a hook-and-loop fastener (38). In some embodiments, during use to hold an endotracheal tube in place, encircling band (30) is separated from chin portion (14) by opening hook-and-loop fasteners (38), removing from loops (34) and discarding. A new encircling band (30) is provided, the ends threaded through loops (34) and the hook-and-loop fasteners (38) closed. Such configuration also allows the effective length of encircling band (30) to be adjusted.

In some embodiments, a strap assembly includes an additional portion (40) configured for encircling a human head under the chin thereof. In some embodiments, such an additional portion is adjustable in length.

In some embodiments, a connector element (16) is movably attached to the strap assembly through a proximal end (42) of the connector element through the chin portion (14) of the strap assembly. In some such embodiments, the connector element is connected to a front of the chin portion of the strap assembly, as depicted in FIG. 1.

In some other embodiments, a transverse rail (90) is attached to the chin portion (14) of the strap assembly, in a direction parallel to a mouth of a patient, namely in a transverse direction. The transverse rail (90) is configured to accommodate the proximal end (42) of the connector element (16) and allow movement of the proximal end (42) in a transverse direction. In one embodiment, the transverse rail (90) is configured allow independent transverse movement of the proximal end (42), without a need of for example moving the proximal end (42) by pushing it by hand. Thus, the proximal end (42) may transversely move in the transverse rail (90) as a result of for example movement of a head of a patient from side to side, without the need of any outside intervention. According to another embodiment, the transverse rail (90) is configured to lock the proximal end (42) in position, and not allow its movement when a slight movement force is exerted on it. According to yet another embodiment, the lockage of the proximal end (42) may be released by exertion of a relatively high movement force on the proximal end (42), for example by forcibly moving the proximal end (42) by hand, or while changing a position of the patient by a caregiver, or by movement of the head by the patient. The principle is that during most of the time the proximal end (42) is locked in position in the transverse rail (90), however when there is a need to move it, it is moved without any outside intervention. This may be achieved, for example, by a transverse rail (90) comprising a plurality of locking elements (92), for example tooth-like structures, slots or clefts, and the like, along the transverse rail (90). Thus, the proximal end (42) is locked in position by a locking element (92), and there is a need to exert force in order to release the lockage, and move the proximal end (42) along the transverse rail (90) until the force is released and the proximal end (42) is locked in position by another locking element (92).

In some embodiments, the connector element is rigid, for example, is fashioned of rigid PTFE, stainless steel or polycarbonate. In some embodiments, by rigid is meant that the connector bends by not more than 0.1 mm when a proximal end thereof is secured and a 1 kg weight is suspended by a distal end thereof In some embodiments, the connector element is semi-rigid, e.g., made of a semi rigid rubber or silicone rubber. In some embodiments, by rigid is meant that the connector bends by not less than 0.1 mm when a proximal end thereof is secured and a 1 kg weight is suspended by a distal end thereof.

In some embodiments, the connector element is straight.

In some embodiments, the connector element is curved, preferably upwardly curved, as depicted in FIG. 1. An (upwardly) curved connector element provides more clearance between the chin and lower lip of a human wearing the holder.

In some embodiments, the connector element is bent (that is to say, has a bent (discontinuous) shape), preferably upwardly bent, for example, an L shape. An (upwardly) bent connector element provides more clearance between the chin and lower lip of a human wearing the holder.

In some embodiments, the distance from the chin portion (14) of the strap assembly to the tube holder attachment (22) is fixed.

In some embodiments, the distance from the chin portion (14) of the strap assembly to the tube holder attachment (22) is not less than 5 cm, not less than 6 cm, not less than 8 cm and even not less than 10 cm. In some embodiments, the distance from the chin portion (14) of the strap assembly to the tube holder attachment (22) is not more than 30 cm.

In some embodiments, the harness is configured so that a distance from the lower lip of a human wearing the harness to the tube holder attachment (22) is not less than 5 cm, not less than 6 cm, not less than 8 cm and even not less than 10 cm. In some embodiments, the harness is configured so that a distance from the lower lip of a human wearing the harness to the tube holder attachment (22) is not more than 30 cm.

In some embodiments, the movable attachment of the connector element (16) to the strap assembly (12) allows rotation of the connector element (16) relative to the chin portion (14) around at least one axis. In some embodiments, the movable attachment of the connector element (16) to the strap assembly (12) allows rotation of the connector element relative to the chin portion around at least two axes, in some embodiments two axes being mutually perpendicular. In some embodiments, the movable attachment of the connector element (16) to the strap assembly (12) allows rotation of the connector element relative to the chin portion around three axes, in some embodiments at least two and in some embodiments all three axes being mutually perpendicular. In some embodiments, such rotation around an axis is in a limited arc, e.g., of not more than 240°, not more than 180°, and even not more than 120°.

In some embodiments, the movable attachment of the connector element (16) to the strap assembly (12) is around a pivot point, as depicted in FIG. 1 where proximal end (42) comprises a flexible silicone rubber knob or a ball joint.

In some embodiments, the tube holder attachment (22) is configured for immovable attachment of an orotracheal tube holder (24).

In some embodiments, the tube holder attachment (22) is configured for movable attachment of an orotracheal tube holder (24). In some such embodiments, the movable attachment allows rotation of an attached orotracheal tube holder around at least one axis relative to the connector element. In some such embodiments, the movable attachment allows rotation of an attached orotracheal tube holder around at least two axes relative to the connector element, in some embodiments the two axes being mutually perpendicular. In some such embodiments, the movable attachment allows rotation of an attached orotracheal tube holder around three axes relative to the connector element, in some embodiments at least two and in some embodiments all three axes being mutually perpendicular. In some embodiments, such rotation is in a limited arc, e.g., of not more than 240°, not more than 180°, and even not more than 120°. In some such embodiments, the movable attachment allows rotation of an attached orotracheal tube holder around a pivot point relative to the connector element, as depicted in FIG. 1 where tube holder attachment (22) comprises a flexible silicone rubber knob or a ball joint.

In some embodiments, a tube holder attachment is configured for attachment to an orotracheal tube holder from beneath the tube holder as depicted in FIG. 1. In some embodiments, such attachment leaves the philtrum of a human wearing the harness unobstructed, as depicted in FIG. 1.

In some embodiments, the harness further comprises an elongated orotracheal tube holder (24) attached to the tube holder attachment (22), the orotracheal tube holder having a distal end (44), a proximal end (46) and an axis (26), the axis directable at the mouth of a human wearing the harness. In some embodiments, the axis is directable through the mouth towards the cervical vertebrae of a human wearing the harness.

In some embodiments, the tube holder (24) is removably attached to the connector element (16) through the tube holder attachment (22).

In some embodiments, the tube holder (24) is irremovably attached to the connector element (16) through the tube holder attachment (22).

In some embodiments, the connector element (16) is removably attached to the strap assembly (12). In some embodiments, the connector element (16) is irremovably attached to the strap assembly (12).

In some embodiments, the tube holder (24) has a length such that the distal end (44) of the tube holder can enter the mouth of a human wearing the harness, see FIG. 1.

In some embodiments, the distal end of the tube holder is bite resistant, that is to say, can withstand the bite of a normal human without deformation of an orotracheal tube held therein.

According to an aspect of some embodiments of the teachings herein, there is also provided an orotracheal tube holder (24), comprising:
an elongated body (48) having a distal end (44) and a proximal end (46);
the distal end (44) being bite resistant and dimensioned to pass into the mouth of a human;
the elongated body defining a void (50) not less than 5 cm long with an axis (26) passing through the void,
the elongated body configured for containing an orotracheal tube (52) inside the void (see FIG. 1). In some embodiments, the orotracheal tube holder comprises an orotracheal tube holder harness as described herein.

The axis passing through the void is an imaginary line passing through the center of an orotracheal tube contained inside the void.

As noted above, the distal end (44) is dimensioned to pass into the mouth of a human. In some embodiments, such dimensioning includes having an outer dimension (e.g., diameter or equivalent) of not less than 0.5 cm but not greater than 2.5 cm.

In some embodiments the void is not less than 8 cm, not less than 10 cm and even not less than 15 cm long. In some embodiments, the void is not more than 30 cm long.

In some embodiments, the length of the void is adjustable (i.e., a user of the tube holder may change the length of the void). In some such embodiments, the tube holder comprises an attachment component (62) configured for attachment of the tube holder to an orotracheal tube holder harness (e.g., an orotracheal tube holder harness according to the teachings herein), and configured to allow the length of the void to be adjustable while keeping a length between the attachment component (62) and the distal end of the elongated body constant. In such embodiments, the distance from the attachment component to the distal end of the elongated body does not change when the length of the void is adjusted.

In some embodiments, the tube holder is configured to hold an orotracheal tube contained inside the void by contacting the orotracheal tube at at least two points along the length thereof, the two points mutually separated by at least 2 cm and/or contacting the orotracheal tube continuously along a length of at least 2 cm (in some embodiments at least 3 cm, at least 4 cm, and even at least 5 cm) of the orotracheal tube, such holding effective to prevent axial slipping of the orotracheal tube contained in the void of the tube holder when the orotracheal tube is deployed for use in a human. In some embodiments, such holding allows maintaining a substantially constant inner diameter of an orotracheal tube held in the void and prevention of kinking of an orotracheal tube held in the void. Orotracheal tube holder (24) depicted in FIG. 1 and in FIG. 2A, contacts an orotracheal tube (54) contained in void (50) continuously along a length of at least 5 cm. Specifically, the substantially half-tube surface of void (50) of tube holder (24) that is defined by component (64) in FIG. 2B has inner dimensions that are substantially the same as the outer dimensions of orotracheal tube (54) so that the contact between tube holder (24) and orotracheal tube (24) is effective to prevent axial slipping of the orotracheal tube contained in the void when the orotracheal tube is deployed for use in a human. However, it is important to note that when tube holder (24) is used to hold a smaller-diameter orotracheal tube, axial slipping is prevented by other components as described below.

In the art, orotracheal tubes comprise a standard respirator connector at the proximal end thereof, a well known standard component typically of a hard plastic with an outer diameter of 15 mm. In some embodiments, a tube holder according to the teachings herein is additionally or alternatively configured to hold an orotracheal tube contained in the void by contact with a respirator connector of the orotracheal tube. In some such embodiments, the proximal end of the elongated body of the tube holder is configured to contact a respirator connector of an orotracheal tube contained in the void, thereby holding the orotracheal tube, such holding effective to prevent axial slipping of the orotracheal tube when the orotracheal tube is deployed for use in a human.

For example, in FIG. 1, the shaft portion (54) of orotracheal tube (52) is contained in void (50) of tube holder (24) and held in place by contact between respirator connector (56) of orotracheal tube (52) and the proximal end (46) of tube holder (24).

In some embodiments, the tube holder has a top side (58) and a bottom side (60).

In some embodiments, on the bottom side of the tube holder is a component (62) configured for attachment of the tube holder to an orotracheal tube holder harness, for example, a harness as described herein. In some embodiments, such an attachment component is not less than 10 cm from a distal tip of the elongated tube holder body, for example, allowing at least a 5 cm clearance from the lower lip while penetrating into the mouth by at least 5 cm, and in some embodiments, not less than 15 cm from a distal tip of the tube holder body.

In some embodiments, the inner dimensions of the void at the distal end of the tube holder are such that allow free passage of an orotracheal tube therein, see FIG. 2A.

In some embodiments, the distal end of the tube holder comprises a wall with a tubular cross section.

In some embodiments, the distal end (44) of the tube holder comprises a wall having a partial tubular cross section (the wall being an incomplete tube with a cross section that is an arc) of not less than 180° (i.e., a cross section that is an arc of not less than a semicircle), not less than 240°, and even not less than 270°. When a person bites down on such a distal end, such configuration allows the distal end to compress somewhat reducing the level of discomfort felt by the person and avoiding damage to the teeth of the person without substantially deforming a held orotracheal tube held, In the embodiment depicted in FIGS. 2, distal end (44) has a partial tubular cross section of 270°.

In some embodiments, the distal end of the tube holder comprises a wall having a cross section with an open lowercase sigma shape, having a partial tubular cross section, where one end of the cross section overhangs the other, such as depicted in FIGS. 2. The overhang limits the extent that the distal end can be compressed to the point where the overhanging end meets the other end.

In some embodiments, the elongated body of the tube holder is made up of at least two separate parts (64, 66/84, see FIGS. 2), mutually movable between at least two states:
an open state allowing placement of an orotracheal tube into the void and removal of the orotracheal tube therefrom; and
a holding state wherein an orotracheal tube is held in the void and aligned with the axis of the void.

In some embodiments, a tube holder is configured, when in the holding state, to contact an orotracheal tube at at least two points (in some embodiments, at least 3, at least 4 and even at least 5 points) mutually separated by at least 2 cm and/or to contact an orotracheal tube continuously along a length of at least 2 cm (in some embodiments at least 3 cm, at least 4 cm, and even at least 5 cm) of the orotracheal tube, such holding effective to prevent axial slipping of an orotracheal tube contained in the void of the tube holder when the orotracheal tube is deployed for use in a human. In some embodiments, such holding allows maintaining a substantially constant inner diameter of an orotracheal tube held in the void and prevention of kinking of an orotracheal tube held in the void.

In some embodiments, a tube holder is additionally or alternatively configured, when in the holding state, to hold an orotracheal tube contained in the void by contact with a respirator connector of the orotracheal tube. In some such embodiments, the proximal end of at least one of the at least two separate parts of the tube holder is configured to contact a respirator connector of an orotracheal tube contained in the void, thereby holding the orotracheal tube, such holding effective to prevent axial slipping of the orotracheal tube when the orotracheal tube is deployed for use in a human.

Tube holder (24) depicted in FIGS. 1 and 2 is configured to hold an orotracheal tube contained in the void (50) by contact with a respirator connector (56) of the orotracheal tube (52). Specifically, proximal end (46) of tube holder (24) is shaped so that the body of a respirator connector (58) rests in trough (68), while the circular flange of a respirator connector (58) presses against proximal face (70) of tube holder (24). Typically, a holding component, such as an elastic band or holder, is wound around proximal end (46) and support flange (72) to keep the respirator connector in place.

In some embodiments, at least two of the at least two separate parts are movable between the open state and the holding state about a hinge. In some such embodiments, the two separate parts are physical distinct but mutually connected by the hinge.

In some embodiments, at least two of the at least two separate parts are mateable to the holding state from an open state, as depicted in FIG. 2. In some such embodiments, at least two of the at least two separate parts are separable from the holding state to the open state, as depicted in FIGS. 2. In some such embodiments, the two separate parts are physical distinct and also entirely separable one from the other.

In some embodiments, in the holding state, the at least two separate parts clamp an orotracheal tube contained in the void, as depicted in FIG. 1.

In some embodiments, in the holding state, at least two of the at least two separate parts intimately encircle an orotracheal tube located in the void, as depicted in FIGS. 1.

In some embodiments, the at least two separate parts comprise a lower part (64) and an upper part (66, 68),
the lower part (64, see FIG. 2B) including the distal end (44), and having a proximal portion (74) shaped as an open-trough; and
the upper part (66, see FIG. 2C or 68, see FIG. 2E) configured to mate with the proximal portion (74) of the lower part (64) when in the holding state.

In some embodiments, on the lower part (64) is a component (50) configured for attachment to an orotracheal tube holder, as seen in FIG. 2A. In some embodiments, on the lower part (64) is a component (62) configured for attachment of the tube holder to an orotracheal tube holder harness, as seen in FIG. 2A. In some embodiments, such a component (62) that is configured for attachment of the tube holder to an orotracheal tube holder harness is not less than 10 cm from a distal tip of the lower part (64), for example, allowing at least a 5 cm clearance from the lower lip while penetrating into the mouth by at least 5 cm.

In some embodiments, at least two of the two separate parts are mateable in at least two different ways, the void of the tube holder being of different length in the at least two different ways. In orotracheal tube holder (24) depicted in FIGS. 2, parts (64) and (66) are mateable in at least two different ways so that the void of the tube holder is of different length in the at least two ways so that the void (50) thereby has an adjustable length.

As seen in FIG. 2B, the portion of void (50) defined by the trough-shape of proximal portion (74) of lower part (64) is flanked by five pairs of identical teeth, neighboring teeth (76) defining four pairs of identical gaps (78).

As seen in FIG. 2C, the edges of the portion of void (50) defined by upper part (66) include five pairs of identical teeth (80) and five pairs of identical gaps (82). Teeth (76) of lower part (64) are dimensioned to fit in gaps (82) of upper part (66). Teeth (80) of upper part (66) are dimensioned to fit in gaps (78) of lower part (64).

When a user mates lower part (64) with upper part (66), bringing tube holder (24) from an open state (FIGS. 2B and 2C) to a holding state (FIG. 2A), the user selects which teeth (76 and 80) are placed in which gaps (82 and 78, respectively), thereby determining the length of tube holder (24) in the holding state. Such configuration allows the length of void (50) to be adjustable while keeping the length between attachment component (62) and the distal tip of tube holder constant. In FIG. 2A, tube holder (24) is in the shortest holding state.

In FIGS. 2D and 2E, an alternative upper part (84) is depicted: in FIG. 2D mated with a lower part (64) identical to the depicted in FIG. 2B and in FIG. 2E alone. The use of upper part is identical to the use of upper part (66) described above. The only substantial difference between upper part (66) described above and upper part (84) is that upper part (84) includes two support flanges (72a and 72b).

Upon carefully perusing the specification and accompanying figures, a person having ordinary skill in the art is capable of implementing the teachings herein using manufacturing methods and materials known in the field of medical devices. For example, some embodiments of a tube holder according to the teachings herein may be manufactured by injection molding of a suitable polymer such as PMMA or silicone rubber.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and scope of the appended claims.

Citation or identification of any reference in this application shall not be construed as an admission that such reference is available as prior art to the invention.

Section headings are used herein to ease understanding of the specification and should not be construed as necessarily limiting.

1. An orotracheal tube holder, comprising:
an elongated body having a distal end and a proximal end;
said distal end being bite resistant and dimensioned to pass into the mouth of a human; and
said elongated body defining a void not less than 5 cm long with an axis passing through said void,
said elongated body configured for containing an orotracheal tube inside the said void.

2. The tube holder of claim 1, comprising an attachment component configured for attachment of the tube holder to an orotracheal tube holder harness, and configured to allow said length of said void to be adjustable, while keeping a length between said attachment component and said distal tip constant.

3. The tube holder of claim 1, configured to hold an orotracheal tube contained inside the void by at least one of:
contacting the orotracheal tube at least two points along the length thereof, the two points mutually separated by at least 2 cm; and
contacting the orotracheal tube continuously along a length of at least 2 cm, such holding effective to prevent axial slipping of the orotracheal tube contained in said void of the tube holder when the orotracheal tube is deployed for use in a human.

4. The tube holder of claim 1, having a top side and a bottom side, and on said bottom side of said tube holder a component configured for attachment of the tube holder to an orotracheal tube holder harness.

5. The tube holder of claim 1, wherein inner dimensions of said void at said distal end are such that allow free passage of an orotracheal tube therein.

6. The tube holder of claim 1, said distal end comprising a wall having a cross section with an open lowercase sigma shape.

7. The tube holder of claim 1, said elongated body made up of at least two separate parts, said two separate parts mutually movable between at least two states:
an open state allowing placement of an orotracheal tube into said void and removal of the orotracheal tube therefrom; and
a holding state wherein an orotracheal tube is held in said void and aligned with said axis of said void.

8. The tube holder of claim 7, at least two of said at least two separate parts being movable between said open state and said holding state about a hinge.

9. The tube holder of claim 7, at least two of said at least two separate parts being mateable to said holding state from said open state.

10. The tube holder of claim 7, said at least two separate parts comprising a lower part and an upper part,
said lower part including said distal end , and having a proximal portion shaped as an open-trough; and
said upper part configured to mate with said proximal portion of said lower part when in said holding state.

11. An orotracheal tube holder harness, configured to be worn by a human, comprising:
a strap assembly including a chin portion, said strap assembly configured for encircling the chin and head and/or neck of a human wearing the harness;
a connector element movably attached to said strap assembly through a proximal portion thereof, so that said connector element extends from said chin portion of said strap assembly; and
a distal portion of said connector element including an orotracheal tube holder attachment, configured for attachment of an elongated orotracheal tube holder having an axis,
wherein, the axis of a tube holder attached to said tube holder attachment of said connector element is directable at a mouth of a human wearing the harness.

12. The harness of claim 11, wherein the axis of a tube holder attached to said tube holder attachment of said connector element is directable through the mouth towards the cervical vertebrae of a human wearing the harness.

13. The harness of claim 11, said strap assembly including an encircling band functionally associated with said chin portion of said strap assembly, together configured to encircle the front of the chin and at least a portion of the cervical spine of a human subject wearing the harness.

14. The harness of claim 11, said encircling band configured to have an adjustable diameter.

15. The harness of claim 11, wherein said strap assembly is configured to allow reversible separation of said strap assembly into at least two parts while an orotracheal tube is held in place in a human subject with the harness:
a first of said at least two parts including said chin portion; and
a second of said at least two parts including other portions of said strap assembly.

16. The harness of claim 11, said strap assembly including an additional portion configured for encircling a human head under the chin thereof.

17. The harness of claim 11, said connector element movably attached to said strap assembly through a proximal end of said connector element through said chin portion of said strap assembly.

18. The harness of claim 11, wherein said connector element is curved.

19. The harness of claim 11, wherein said connector element is bent.

20. The harness of claim 11, wherein a distance from said chin portion of said strap assembly to said tube holder attachment is fixed.
